(19) United States
(12) Patent Application Publication
(10) Pub. No.: US 2018/0092983 A1
(43) Pub. Date: Apr. 5, 2018

(54) METHODS AND REAGENTS TO TREAT AUTOIMMUNE DISEASES AND ALLERGY

(71) Applicant: Tian Xin Wang, Walnut Creek, CA (US)

(72) Inventor: Tian Xin Wang, Walnut Creek, CA (US)

(73) Assignee: Tian Xin Wang, Walnut Creek, CA (US)

(21) Appl. No.: 15/723,173

(22) Filed: Oct. 3, 2017

Related U.S. Application Data
(60) Provisional application No. 62/404,204, filed on Oct. 5, 2016.
(60) Provisional application No. 62/470,338, filed on Mar. 13, 2017.

(51) Int. Cl. A61K 47/64; A61K 9/70; A61K 39/00; A61K 47/55

(52) U.S. Cl. CPC A61K 47/646; A61K 9/7023; A61K 2039/60; A61K 47/55; A61K 2039/54; A61K 39/0008

(57) ABSTRACT

The present invention provides compositions for inducing immune tolerance and methods to modify antigen to treat disease such as autoimmune diseases and allergy. Disclosed are compositions, and related methods, comprising APC presentable antigens and immunosuppressants that provide tolerogenic immune responses specific to antigen. In some embodiments, the composition is used for transdermal delivery.

### CROSS-REFERENCE TO RELATED APPLICATIONS

This application claims priority to U.S. Provisional Patent Application 62/404,204 filed on Oct. 5, 2016 and U.S. Provisional Patent Application 62/470,338 filed on Mar. 13, 2017. The entire disclosure of the prior application is considered to be part of the disclosure of the instant application and is hereby incorporated by reference.

### BACKGROUND OF THE INVENTION

### Field of the Invention

The current invention relates to protein, peptide and antigen modification for pharmaceutical applications and reagents to treat disease such as auto immune disease and allergy. The current invention discloses methods to treat auto immune disease and allergy. The current invention also relates to methods to treat auto immune disease with cell based therapy.

### Background Information

Immune responses are necessary for protection against potentially pathogenic microorganisms. However, undesired immune activation can cause injurious processes leading to damage or destruction of one's own tissues. Undesired immune activation occurs, for example, in autoimmune diseases where antibodies and/or T lymphocytes react with self antigens to the detriment of the body's tissues. This is also the case in allergic reactions characterized by an exaggerated immune response to certain environmental matters and which may result in inflammatory responses leading to tissue destruction. This is also the case in rejection of transplanted organs which is significantly mediated by alloreactive T cells present in the host which recognize donor alloantigens or xenoantigens. Immune tolerance is the acquired lack of specific immune responsiveness to an antigen to which an immune response would normally occur. Typically, to induce tolerance, there must be an exposure to a tolerizing antigen, which results in the death or functional inactivation of certain lymphocytes. This process generally accounts for tolerance to self antigens, or self-tolerance. Immunosuppressive agents are useful in prevention or reduction of undesired immune responses, e.g., in treating patients with autoimmune diseases or with allogeneic transplants. Conventional strategies for generating immunosuppression associated with an undesired immune response are based on broad-acting immunosuppressive drugs. Additionally, in order to maintain immunosuppression, immunosuppressant drug therapy is generally a life-long proposition. Unfortunately, the use of broad-acting immunosuppressants is associated with a risk of severe side effects, such as tumors, infections, nephrotoxicity and metabolic disorders. Accordingly, new immunosuppressant therapies would be beneficial.

Extracorporeal therapy is a procedure in which blood is taken from a patient's circulation to have a process applied to it before it is returned to the circulation. All of the apparatus carrying the blood outside the body is termed the extracorporeal circuit. It includes hemodialysis, hemofiltration, plasmapheresis, apheresis and etc. Plasmapheresis is the removal, treatment, and return of (components of) blood plasma from blood circulation. The procedure is used to treat a variety of disorders, including those of the immune system, such as myasthenia gravis, lupus, and thrombotic thrombocytopenic purpura. Hemoperfusion/hemopurification is a medical process used to remove toxic or unwanted substances from a patient's blood. Typically, the technique involves passing large volumes of blood over an adsorbent substance. The adsorbent substances most commonly used in hemoperfusion/hemopurification are resins and activated carbon. Hemoperfusion/hemopurification is an extracorporeal form of treatment because the blood is pumped through a device outside the patient's body. Its major uses include removing drugs or poisons from the blood in emergency situations, removing waste products from the blood in patients with renal failure, and as a supportive treatment for patients before and after liver transplantation. Apheresis is a medical technology in which the blood of a donor or patient is passed through an apparatus that separates out one particular constituent and returns the remainder to the circulation. Depending on the substance that is being removed, different processes are employed in apheresis.

### BRIEF DESCRIPTION OF THE DRAWINGS

FIG. 1 shows an example of ADC for SLE treatment

FIG. 2 shows example of general structure of Epitope(antigen)-alpha gal conjugate

FIG. 3 shows an example of antigen-alpha gal conjugate for SLE treatment

FIG. 4 shows examples of antigen-cell inactivating molecule conjugate

FIG. 5 shows examples of VEGF-cell inactivating molecule conjugate for cancer treatment

FIG. 6 shows example of FITC-DNA auto antigen conjugate to treat lupus by inactivating the DNA specific auto antibody producing B cells.

FIG. 7 shows the examples of drug conjugated to carbohydrate polymer to form prodrug.

FIG. 8 shows an example of polysialic acid conjugated with rapamycin with ester bond to form a prodrug.

FIG. 9 shows an example in which the linker is a glycine to connect drug and polymer backbone.

FIG. 10 shows an example of hyaluronic acid based methotrexate prodrug.

FIG. 11 shows an example of the structure of the conjugate containing both antigen and rapamycin.

FIG. 12 shows examples of 3 different formats of the antigen-drug conjugate.

FIG. 13 shows examples of an epitope(antigen)-sialic acid rich polymer conjugate to treat autoimmune disease or allergy or to induce immune tolerance.

FIG. 14 shows examples of the conjugate containing antigen and sialic acid/siglec ligand.

FIG. 15 shows schematic example of the structure of the microsphere based agent to induce immune tolerance and treating auto immune diseases or allergy.

FIG. 16 shows different formats of using polymer carrier conjugated with antigen, siglec ligand and other immunosuppressant; and both siglec ligand and other immunosuppressant conjugated to the antigen.

FIG. 17 shows examples of siglec ligand-antigen conjugate for systemic lupus erythematosus treatment.

### DESCRIPTION OF THE INVENTIONS AND THE PREFERRED EMBODIMENT

In one aspect, the current invention discloses a transdermal drug delivery system such as a transdermal patch to treat conditions selected from autoimmune disease, allergy and anti-drug antibody comprising an antigen causing said condition and an immunosuppressant. The antigen can be B cell antigen, T cell antigen in MHC-peptide complex form or the antigen peptide (or its derivative) of T cell antigen that can bind with MHC to form the MHC-peptide complex. Example of immunosuppressant is selected from rapamycin, fujimycin and methotrexate. The current invention also discloses a method to treat autoimmune disease or allergy or inhibit anti-drug antibody production or induce antigen specific immune tolerance in a subject by administering to the subject a said transdermal drug delivery system on the skin.

In another aspect, the current invention discloses a conjugate to treat conditions selected from autoimmune disease, allergy and anti-drug antibody comprising an antigen causing the condition, a first immunosuppressant and a second immunosuppressant. The antigen can be B cell antigen, T cell antigen in MHC-peptide complex form or the antigen peptide (or its derivative) that can bind with MHC. The first immunosuppressant is selected from siglec ligand such as sialic acid or poly sialic acid. Example of second immunosuppressant is selected from rapamycin, fujimycin and methotrexate. The current invention also discloses a method to treat autoimmune disease or allergy or inhibit anti-drug antibody production or induce antigen specific immune tolerance in a subject by administering to the subject the said conjugate (e.g. subcutaneous or intravenous injection).

In one aspect of the current inventions, solid phase support adsorbent with auto antigen coated on the surface can be used in hemopurification to remove the autoimmune T cell or B cell from the patient's blood to treat their auto immune disease, similar to removing the CTC from the patient to treat cancer described the previous U.S. patent application Ser. No. 13/444,201 and U.S. Ser. No. 15/373,483. For example a hemopurifier with adsorbent (e.g. particles) coated with insulin and/or bata cell surface antigen can be used to remove auto immune T cell/B cell clones to treat diabetes. One can also separate the Lymphocyte from the blood with blood cell separator/leukapheresis and then pass the separated lymphocyte through an affinity column (surface coated with auto antigen) or mix with magnetic particles (surface coated with auto antigen) to remove the autoimmune T cell or B cells and then return the blood/lymphocyte back to the patient. The procedure is similar to the CTC removal described in the U.S. Ser. No. 13/444,201 and U.S. Ser. No. 15/373,483 applications by the current inventor except the target is B cell or T cells having affinity to certain auto antigens. The current invention discloses the method of T cell and B cell removal with hemopurification to treat the diseases caused by these T cell and/or B cell clones. The U.S. patent application Ser. No. 13/444,201 by the inventor of the current application disclose hemopurification method, device and reagent to remove auto antibody from blood of the patient using hemopurification cartridge containing affinity matrix coated with antigen specific to the auto antibody. The said hemopurification method, device and reagent can be further applied to the whole blood of the patient to remove the T cell and B cell in the blood that are specific to the coated antigen, therefore to treat the immune disease caused by these T cell/B cell clones in the patient. For example, the said patent application PCT/US2012/033153 (U.S. application Ser. No. 13/444,201) described method, device and reagent to remove CTC from blood using affinity matrix coated with antibody against CTC, when the affinity matrix is coated with pancreatic islet antigen instead, the corresponding method, device and reagent can be used to remove circulating T cells against pancreatic islet therefore to treat diabetes. In another example, the affinity matrix is coated with double strand DNA (e.g. those described in the current invention to conjugate with toxin or alpha-gal), the resulting hemopurification method and device can be used to remove auto immune B cells against DNA, therefore can be used to treat lupus. The antigen can be either B cell antigen or T cell antigen (MHC-peptide complex such as those used for MHC tetramer technology, the MHC and the peptide can be covalently conjugated).

The solid phase support for blood purification could be a column, a membrane, a fiber, a particle, or any other appropriate surface, which contains appropriate surface properties (including the surface of inside the porous structure) either for direct coupling of the affinity molecules or for coupling after modification or for surface derivatization/modification. If the solid support is porous, its inside can also be used to present the binding affinity molecules.

The current invention also discloses antigen-drug conjugate or antigen-alpha gal conjugate for autoimmune disease. The U.S. patent application Ser. No. 13/444,201 discloses methods to treat autoimmune disease/diseases caused by the production of certain antibody or auto immune T cell against certain foreign antigen or auto antigen. The method involves two steps, in the first step; antibodies or specific antibody or B cells/T cells causing the disease is removed by blood purification procedure. Alternatively, instead of using blood purification, production of antibodies or specific antibody causing the disease is inhibited with drugs. Suitable drug includes those can inhibit the production of antibodies such as adrenal corticosteroids, cyclosporin, rapamycin, methotrexate and cellcept. Preferably the dosage is enough to inhibit at least 50% antibody production. The second step is the same as those described in the U.S. Ser. No. 13/444,201 application. When the toxin/cell inhibitor/inactivator-antigen conjugate (e.g. hot suicide antigen or immunosuppressive drug such as those described in “Immunosuppressive drug” article page in Wikipedia) is used to inactivate the antibody production and/or T cells in the second step (e.g. by injecting the conjugate to the object having autoimmune disease caused by the said auto antigen), the epitope of the antigen need to be selected to be those only bind with specific B cell/T cell/antibody but not other receptors in the body. For example, some diabetes is due to the production of insulin antibody, one can use an insulin epitope-toxin conjugate to inactivate the B cell producing insulin antibody by injecting it to the patient at therapeutic effective amount. This epitope need to be selected to only bind with the B cell/T cell/antibody but not the insulin receptor on other human cells.

Many major diseases are caused by auto-antibody (e.g. rheumatoid arthritis and certain diabetes) or bad antibody (e.g. allergy, transplant rejection). Current treatment can not cure from the root and often result in serious side effects (e.g. steroid). ADC (antibody-drug conjugate) becomes a promising cancer treatment in recent years. Antigen-drug conjugate strategy can be used for auto antibody induced autoimmune diseases; selectively inactivate the specific antibody producing B cell clone to cure from the source. The principle was described in U.S. patent application Ser. No. 13/444,201 by the inventor of the current application.

Among billions of B cell clones, only several B cell clones produce specific antibody against certain antigen; these B cells secret monoclonal antibody and present membrane bound antibody (BCR receptor) highly specific for target antigen. Antigen-drug conjugate will bind with these B cells with high affinity/high specificity and inactivate them. Selectively inactivating these B cell clones will eliminate the production of harmful antibodies for treating many auto-antibody induced diseases, e.g. lupus, recurrent fetal loss, rheumatoid arthritis, type 1 diabetes, deep vein thrombosis, myasthenia gravis and more.

Companion test (ELISA) to be performed to identify patient having auto antibodies specific to the ADC (similar to the HER2 test for Herceptin): reducing off target. Hemopurification (a clinically used treatment method) using affinity column immobilized with antigen to remove abundant circulating auto-antibodies: one-time treatment before ADC administration to improve the ADC efficacy/selectivity for B cells. In most cases no need for protein conjugation, peptide epitope or small molecule antigen will be sufficient for ADC construction, simplify the development/manufacture of ADC. Monthly dosing will be sufficient to prevent somatic hypermutation. T cells also present T cell receptor specific to target antigen, inactivating these T cell clones using antigen-drug conjugate may also be used to treat T-cell-mediated autoimmunity in many major diseases.

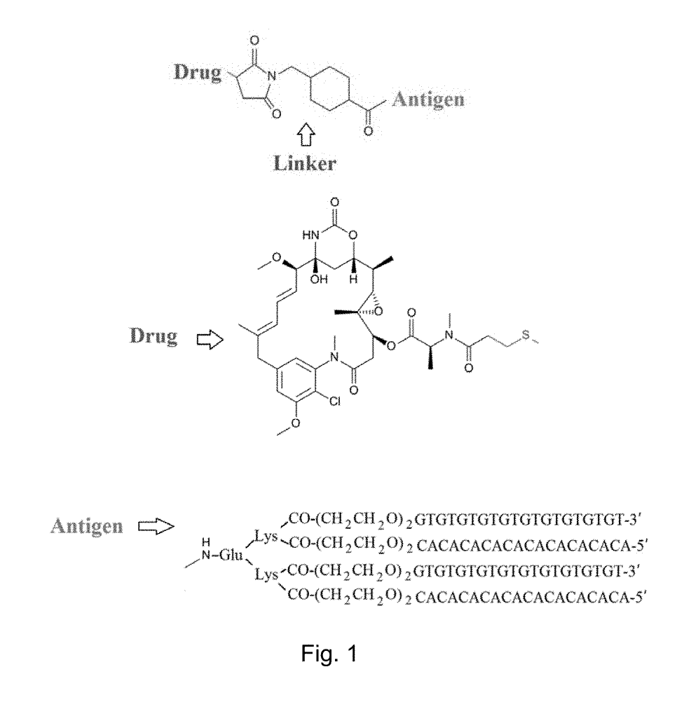

Auto antibody against DNA is a key pathogenic factor in SLE, DNA coated affinity column is clinically used to remove these Ab from patient blood (hemopurification) as an effective SLE treatment. Antigen-drug conjugate can be used for SLE treatment. As shown in FIG. 1, DNA-linker-Mertansine (DNA sequence adopted from Abetimus, linker/toxin adopted from Kadcyla, linker can be optimized for B/T cells) is an example of ADC for SLE treatment. The DNA sequence used are the complex formed with GTGTGTGTGTGTGTGTGTGT (SEQ ID NO: 1) and CACACACACACACACACACA (SEQ ID NO: 2). Single strand DNA antigen can also be used to inactivate auto antibody generating cells specific to single strand DNA. It will selectively inactivate the specific B cell clone producing auto antibody against DNA, treat the disease from the source. It can be prepared easily with solid phase synthesis. It can be intravenously injected to the patient having SLE to treat it. Companion test will be performed to increase the efficacy. Patient will be treated with hemopurification to remove the anti-DNA antibody before the first dose ADC administration for better therapeutical index.

In some embodiments preferably the antigen should not bind with the endogenous receptor, for example, insulin fragment that does not bind with insulin receptor but can bind with insulin auto antibody can be used.

Instead of epitope(antigen)-toxin described in the current application and the previous U.S. application Ser. No. 13/444,201, epitope (antigen)-alpha gal (e.g. Galactose-alpha-1,3-galactose) can also be used instead, which utilize the endogenous anti gal antibody to inactivate the B cell clone or T cell clone that can selectively bind with the epitope (antigen). The alpha gal can be readily adopted from U.S. patent application Ser. No. 12/450,384 and other publication.

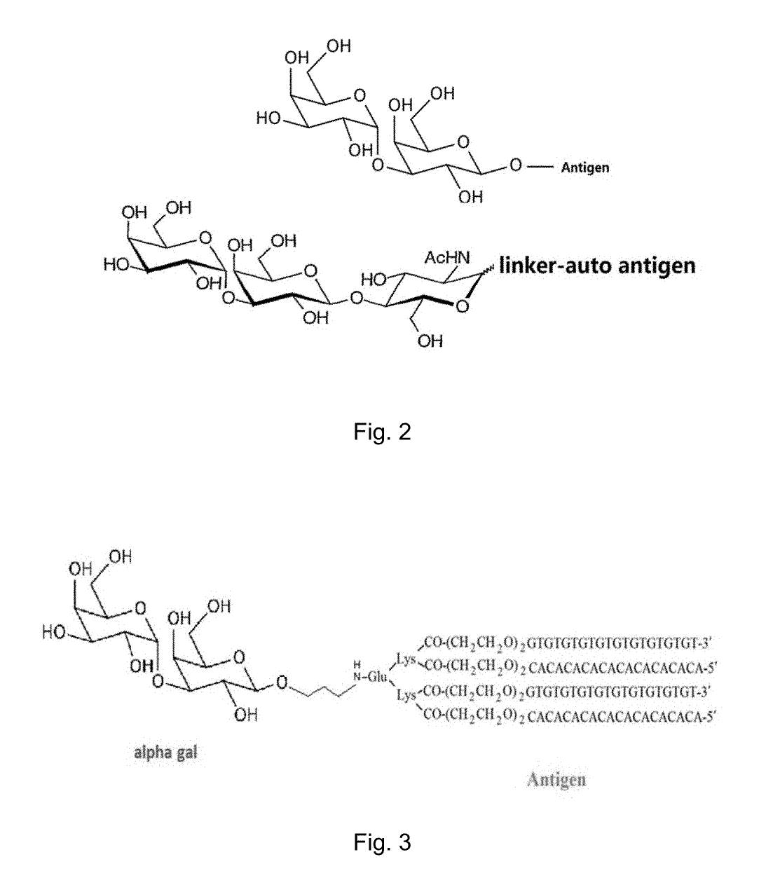

Epitope (antigen)-alpha gal conjugate design has the formula: alpha galactosyl-(optional linker)-epitope (antigen), which will allow the T cell/B cell specific to the epitope (antigen) bind with endogenous anti-Gal antibody and therefore be eliminated/inactivated due to the bound antibody. Examples are shown in FIG. 2.

For example, the antigen can be insulin or insulin fragment that recognized by autoimmune B cell/T cell, or peptide of pancreatic islets recognized by the auto immune T cell in diabetics or the auto antigen of beta cells (e.g. those described in Clin Immunol. 2004 October; 113(1):29-37 and Proc Natl Acad Sci U S A. 2003 Jul. 8; 100(14): 8384-8388). This conjugate will selectively inactive the autoimmune B cell/T cells causing diabetics. For T cell antigen, it can be the MHC-peptide complex form, in which the peptide can be optionally covalently linked with the MHC.

The T cell recognize T cell antigen by its TCR receptor. The T cell antigen normally is in the form of MHC-epitope binding complex. The epitope normally is a peptide (sometimes other molecules such as carbohydrate) processed by APC. In some embodiments of the current invention, the antigen for T cells preferably is the formed MHC-epitope complex or its fragment/derivatives/mimics, which has higher specific affinity to TCR than the epitope alone. It can be the monomer form or oligomer (dimer, trimer, tetramer, pentamer or even higher degree polymer) form such as the MHC tetramer currently widely used in research. For example, HLA-A2insB10-18 tetramer (doi: 10.1073/pnas.0508621102) can be conjugated with the cell inactivating agent with an optional linker to treat Type 1 diabetes by inactivating the auto immune T cell. The epitope (e.g. peptide) can be covalently conjugated with MHC to increase its stability by well known means as disclosed in well known publications. Similarly, the antigen used for B cell in the current invention can also be oligomer or polymer form. However the antigen used for B cell inactivation does not require the MHC component.

An example drug that can selectively inactivate B cells producing auto antibody against DNA is shown in FIG. 3, this drug can be used to treat lupus. The patient can receive 500 mg˜1 g of the said conjugate as weekly i.v. injection to treat his lupus until symptom disappear.

Alternatively, tregitope peptide-antigen conjugate can be used instead of toxin-antigen conjugate for the same purpose. It will selectively inactivate the autoimmune T cell, therefore treat the corresponding diseases.

The carrier system can be used for the above invention as disclosed in U.S. application Ser. No. 13/444,201 by the current inventor. For example, the liposome or microparticle or nanoparticle can be used. The antigen is immobilized on the surface of the liposome or particles and the effector molecule (e.g. alpha gal, rhamnose, immune suppression cytokine, tregitope peptide, toxin, Si RNA or mi RNA or the like, immune suppressant, antisense molecule) can be either encapsulated inside or co-immobilized on the surface of liposome or particles.

Solid phase support adsorbent with auto antigen or combinations of different auto antigens for the same diseases (because sometimes a patient will have T cells/B cells specific for a groups of different auto antigens) coated on their surface can be used in hemopurification to remove the autoimmune T cell or B cell from the patient's blood to treat their corresponding auto immune disease, similar to remove the CTC from the patient to treat cancer (e.g. For example a hemopurifier with adsorbent coated with insulin and/or bata cell surface antigen either alone or their peptide in complex with MHC can be used to remove auto immune B cell or T cell clones to treat diabetes. The method, procedure is similar to the CTC removal described in the current and previous applications by the current inventor except the target is B cell or T cells having affinity to certain auto antigens. The auto antigen for T cell removal is preferably the complex of MHC-epitope as previously described. Because for a specific auto immune diseases sometimes multiple auto antigens are involved (e.g. GAD65, insulin, preproinsulin and etc. for type 1 diabetics), therefore the solid phase support adsorbent can be a mixture of different solid phase support adsorbent each coated with different auto antigen for this diseases; or a solid phase support adsorbent coated with a mixture of the different auto antigen involved for the diseases, similar to the strategy for sepsis treatment described above. An ELISA test can be performed to the patient to identify the antigens involved and use this information to select suitable solid phase adsorbent for treatment.

When the solid phase support (for example those described in the current inventions and previous applications by the applicant, e.g. 100 um˜2 mm microparticles or 50 nm˜100 um magnetic particles) is coated with MHC-epitope complex (either in monomer or oligomer or polymer form, the complex can be either covalent or non-covalent), it can be used to remove T cells against this auto antigen (MHC-epitope complex such as HLA-A2insB10-18) from blood either in hemopurification or blood/blood fragment collected from the patient. The T cells will include a mixture of regulatory T cells and effector T cells/cytotoxic T cells and helper T cells specific to this auto antigen (e.g. HLA-A2insB10-18 for diabetic patient). The effector T cells/cytotoxic T cells can be removed from the mixture by its surface maker CD+ with positive selection method such as well know means such as magnetic beads/flow cytometry/affinity columns. The Treg can be purified/isolated based on its surface maker with well known method such as those described in the publications and the commercial kits (e.g. those described in http://www.bdbiosciences.com/us/applications/research/t-cell-immunology/regulatory-t-cells/m/745680/workflow/tregenrichment). The Treg can also be prepared by in vitro conversion by introducing FOXP3 expression into the cells (e.g. described in Molecular Therapy (2007) 16 1, 194-202). The prepared/purified Treg can be in vitro expanded and optionally further purified again, and then injected back to the patient to treat corresponding auto immune disease. This resulting Treg is antigen specific, therefore provide better efficacy and lower off target effect for the target disease treatment (e.g. HLA-A2insB10-18 for diabetic patient). This method both removes the effector T cells and increase Treg cells in the patient for specific antigen therefore provide better treatment effect for the auto immune disease caused by the said antigen. The mixture of Treg specific to different auto antigens involved in a specific disease can be prepared and injected to the patient having auto immunity to these auto antigens. The Treg can also inhibit the corresponding B cells to inhibit the auto antibody production. Sometimes the T cell antigen is derived from that B cell antigen by APC therefore the Treg can also be used to treat auto immunity generated by auto immune B cell/auto antibody.

Instead of alpha gal, other molecule/peptide/protein can also be used to conjugate with a specific antigen to selectively inactivate the specific B cell clone or T cell clone that binds and reacts with the specific antigen. The resulting agent has the general structure:

### Cell Inactivating Molecule-Linker (Optional)-Antigen

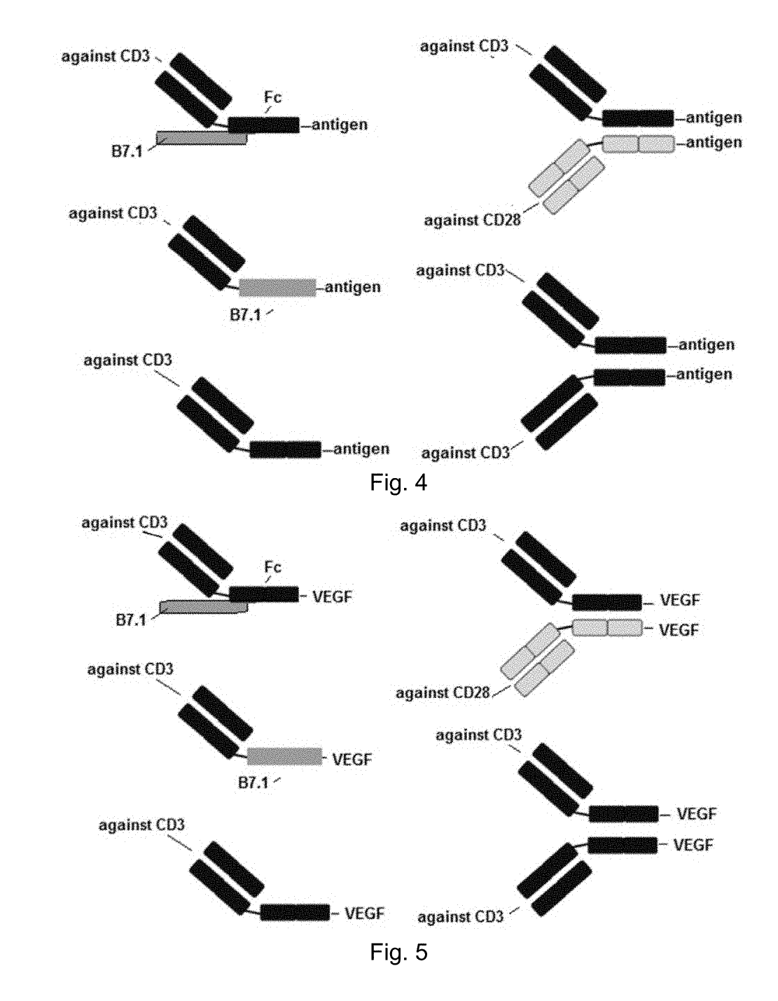

The agent can be given to the patient (e.g. by i.v. injection) at therapeutic effective amount and in therapeutic acceptable formulation to the patient having autoimmune disease or allergy due to the said antigen to treat said autoimmune disease or allergy. When the antigen is a therapeutic drug (e.g. recombinant protein) or its epitope, it can be given to the patient (e.g. by i.v. injection) to inhibit/prevent the production of anti drug antibody (ADA). It can be used to induce antigen specific immune tolerance. Example of cell inactivating molecule include affinity ligand (e.g. antibody or its fragment, aptamer) or their combination against immune cells (e.g. those used in bi specific antibody and triomab for cancer treatment) such as a antibody against a T-lymphocyte antigen like CD3, or a bi specific antibody (or a triomab having Fc) against CD3 and CD28, or a fusion protein of B7 with an antibody (or its fragment) against CD3 (examples shown in FIG. 4), antigen that already has immuno response in the body (e.g. alpha-gal, L-rhamnose), B7, super antigen (e.g. staphylococcal enterotoxin A, SEA), cytokines (e.g. immuno cell inactivating cytokines) and those described in the previous patent applications by the inventor and references. For example, L-rhamnose can be linked with a PEG₃ by a glycoside bond and the PEG₃ is also conjugated with an auto antigen.

When affinity ligand such as antibody or its fragment against cytotoxic immune cell activating receptor such as CD3 of T cell or CD16 of NK cell is conjugated with antigen, it will recruit/activate cytotoxic immune cell such as T cells or NK cells to inhibit/kill the target B/T cell that can bind with the antigen (preferably the antigen for target T cell will be MHC-peptide complex recognized by its TCR); which is similar to the current bi-specific antibody to kill cancer cell except the auto antigen is used in the conjugate instead of the antibody against cancer cell).

For example, in one example an antibody or Fab against CD16A of NK cell (which sequence can be adopted from the TnadAb AFM13 of Affimed) is conjugated with the linker-antigen for SLE shown in FIG. 1 via its cysteine to form a thiol-maleimide linkage, which is widely used in antibody drug conjugate and the conjugation protocol is well known to the skilled in the art. This antigen-anti CD16A antibody conjugate can be used to treat SLE. Once being injected to the patient (e.g. 200 mg˜1000 mg i.v. bi weekly), it will bind with DNA antigen specific B cells and attract NK cell to kill it, therefore inhibit auto antibody production against DNA antigen. Alternatively, an antibody or Fab against CD3 can be used instead of those against CD16 to prepare the conjugate. The resulting conjugate can attract cytotoxic T cell to kill the antigen specific B cell to treat corresponding autoimmune diseases.

Optionally additional affinity ligand can also be introduced into the conjugate to increase the affinity and specificity to B or T cell. For example, antibody against CD20 can also be incorporated in the conjugate via a linker to increase the targeting toward B cell, a scheme similar to tri-specific antibody.

Another type of cell inactivating molecule is SEA. SEA is a microbial super-antigen that activates T-lymphocytes and induces production of various cytokines, including interferon-gamma (IFN-gamma), tumor necrosis factor-alpha (TNF-alpha), and cytolytic pore-forming perforin and/or granzyme B secreted by intratumoral CTLs. Example of the SEA gene utilized here can carry the D227A mutation created by Dohlsten's group, which showed a 1000-fold reduction of binding to major histocompatibility complex class (MHC) II in order to decrease systemic toxicity. The protocol of preparing SEA-conjugate can be found at patent applications CN102114239A, CN1629194A and CN101829322A.

Besides the co-stimulatory molecules B7.1, other co-stimulatory molecules can also be used as cell inactivating molecule such as those selected from other B7 family members including B7.2 (CD86), B7-H1 (PD-L1), B7-H2 (B7RP-1 or ICOS-L or B7h or GL-50), B7-H3 (B7RP-2), B7-H4 (B7x or B7S1), B7-DC (PD-L2) and etc., and these proteins having amino acid sequence of more than 70% identity of the natural and man-made variants. Co-stimulatory molecules B7.1 (CD80) or other co-stimulatory molecule's role is to stimulate the body's immune response. Furthermore, in addition to B7 family members, other molecules can stimulate T cells can also be used as cell inactivating molecule of the present invention. The protocol described in patent application CN102391377A (CN201110338886) can be readily adopted for the current invention. For example, the cytokine of the fusion protein in CN102391377A can be replaced with the auto antigen to generate the conjugate of the current application to inactivate the antigen specific B cell and/or T cells.

B7 is a type of peripheral membrane protein found on activated antigen presenting cells (APC) that, when paired with either a CD28 or CD152 (CTLA-4) surface protein on a T cell, can produce a costimulatory signal or a coinhibitory signal to enhance or decrease the activity of a MHC-TCR signal between the APC and the T cell, respectively. Some type B7 proteins can enhance the activity of T cells (e.g. B7.1, B7.2) and some of them can inhibit the activity of B/T cells (B7.DC/PD-L2, B7.H1/PD-L1). When T cell activating B7 is conjugated with antigen, it will recruit/activate other T cells or cytotoxic immune cells to inhibit/kill (similar to the current bi-specific antibody to kill cancer cell except the auto antigen is used instead of the antibody against cancer cell) the target B/T cell that can bind with the antigen (preferably the antigen for target T cell will be MHC-peptide complex recognized by its TCR). When B/T cell inhibiting B7 is used in the conjugate, it will bind with the corresponding receptors on target B/T cell to kill/inactivate the target B/T cells that can bind with the antigen.

Like B7, other ligand that can activate the inhibitory immune checkpoint receptors on immune cells such as A2AR, B7-H3, B7-H4, BTL, IDO, KIR, LAG3, PD-1, TIM-3 and VISTA, or the ligand (e.g. antibody or its fragment) that can block the activating checkpoint molecules on immune cells such as CD27, CD 28, CD40, CD122, CD137, OX40, GITR, CD52 and ICOS, can also be used as cell inactivating molecule. For example the cell inactivating molecule can be PD-L1 or its derivative/fragment or mimic or other ligand that binds to PD-1 to prevent B or T cell activation, PD-L2 or its derivative/fragment or mimic or other ligand that binds to PD-1 to prevent B or T cell activation and etc.

When the target cell is B cell, BCR antigen-TCR antigen conjugate can also be used. Optional linker can be added between these functional groups. In the conjugate the B cell antigen binds with the target B cell and the T cell antigen (MHC-antigen peptide complex, which can be covalently linked together) bind with the effector T cell. The antigen for B cell and T cell can be different. The principle is to recruit the existing effector T cell to kill/inactivate the target B cell. The T cell antigen can also be the peptide that can bind with the MHC to form the MHC-peptide complex or its derivative, instead of the full MHC-peptide complex type T cell antigen, in this case the peptide will be taken by APC and then form the MHC-peptide complex in vivo to induce immune tolerance.

When the antigen in the conjugate described above and in FIG. 4 is replaced with affinity ligand for cancer cells (e.g. antibody against cancer cell or cytokine/peptide/protein having affinity to cancer cells described in paragraph below), it can be used to treat cancer (examples shown in FIG. 5, the VEGF can be VEGF antagonist such as VEGF165b, the VEGF can also be replaced with an antibody or its fragment against cancer cell). The current invention also discloses methods and agents to treat cancer and kill cancer cells. CN101829322A (PCT/CN2010/078854) discloses the use of a cytokine-superantigen fusion protein for preparing a medicament against cancer/tumor, wherein the cytokine is an epidermal growth factor or a vascular endothelial cell growth factor, and the superantigen is the superantigen of staphylococcus aureus enterotoxin A. SEA-conjugates that can be used to treat cancer are also disclosed at patent applications CN102114239A, CN1629194A and CN101829322A. Superantigen fusion protein for anti-cancer therapy and methods for the production is also disclosed at CN1629194A. Patent application CN102391377A, U.S. Ser. No. 10/571,836. CN102391377A discloses a cancer induction and activation of T cells to target the fusion protein and preparation method and use, the protein comprises a peptide with cancer cells and costimulatory molecules B7.1, the cancer cells with a peptide selected Since TGF-α, epidermal growth factor, vascular endothelial growth factor, or gonadotropin-releasing hormone gastrin-releasing peptide, fusion proteins of the invention has a cancer targeting end, e.g. targeting VEGFR, EGFR, GnRH-R, or GRP-R action, on another end targeting the CD28 receptors expressed on T cells, so it will target T cells to cancer cell or tumor region with highly expressed VEGFR, EGFR, GnRH-R, GRP-R, experiments show that the fusion proteins of the invention can inhibit tumor growth and induces apoptosis of cancer cells. The patents listed above utilize B7.1 or super antigen conjugated with a cytokine or peptide or protein that can bind with cancer cell.

The current invention also disclose a method and agent to treat cancer and kill cancer cells by conjugate the cytokine or peptide or protein used in the above patents (which was conjugated to B7 or super antigen) with alpha gal or antibody that can bind with immune cells (such as those used in the bispecific antibody for cancer treatment, e.g. antibody against a T-lymphocyte antigen like CD3). Administering the resulting conjugate to the patient can be used to treat cancer. Several examples of the conjugate are: alpha gal-linker (optional)-EGF, alpha gal-linker(optional)-VEGF, alpha gal-linker(optional)-TGF-α, alpha gal-GnRH. Preferably the resulting conjugate does not have EGFR/VEGFR agonist activity. When native EGF or VEGFR is used, the conjugate may still have agonist activity. Preferably affinity ligand that can bind with EGFR or VEGFR without activating them, e.g. EGFR or VEGF antagonist, is used to prepare the conjugate. For example, decorin, VEGF165b, VEGF antagonist in PCT/CA2010/000275 can be used to prepare the conjugate instead of using native VEGF that can activate VEGFR for angiogenesis; they can also be used to conjugate with toxin (such as MMAE, MMAF and DM1) for cancer treatment. These cytokines can be further modified to be peptidase/protease resistant to increase their half life in vivo and a half life modifier such as Fc or fatty acid can be added into the conjugate to increase their half life.

The conjugate of alpha gal or L-rhamnose with peptide/protein/small molecules (e.g. folic acid, VEGF or their derivatives/mimics such as VEGF165b and those disclosed previously) that can bind with cancer cells can also be used to treat cancer. Examples of them include folic acid-optional linker-alpha gal, VEGF165b-optional linker-alpha gal, VEGF-optional linker-alpha gal, folic acid-optional linker-alpha L-rhamnose, VEGF165b-optional linker-alpha L-rhamnose, VEGF-optional linker-alpha-L-rhamnose.

Besides alpha gal, other antigen that already has T cell immunity or B cell immunity can also be used to replace the alpha gal in the said conjugate for immune cell or cancer cell or pathogen inactivation. It can be either endogenous or induced by vaccination using the said antigen. Examples of endogenous antigen include DNP (Dinitrophenyl) and L-rhamnose (e.g. alpha-L-rhamnose). The induced antibody or antigen specific effector T cell can be generated with vaccination. For example, most new born receive the antituberculosis vaccine BCG, the oral poliovirus vaccine (OPV) and the anti-hepatitis B vaccine (HBVac). They will have B cell or T cell immunity against these antigens. One can use the antigen from OPV or BCG or HBV to prepare the conjugate instead of using alpha gal. The patient can be first tested with his antigen reactivity and select the antigen having strong B cell or T cell immunity to prepare the conjugate and administering this personalized conjugate to the patient to treat his diseases (e.g. cancer or auto immune disease). One can also inject the patient with a vaccine for a special antigen (e.g. a non-native peptide antigen conjugated to KLH, administrated with boosters) to allow the patient to develop T cell immunity or B cell immunity against this antigen and then use this antigen to prepare the conjugate described in the current invention for disease treatment. Another example of utilizing native immunity is to use the blood type antigen instead of alpha-gal to build the conjugate: ABO antigen. For example, for patient having Blood type group A, the conjugate can utilize B antigen; for patient having Blood type group the conjugate can utilize A antigen; for patient having Blood type group O, the conjugate can utilize either A or B antigen or their combination. In one example, the conjugate of A antigen-double strand DNA can be used to treat blood type B patient having lupus; in another example, the conjugate of B antigen-VEGF165b can be used to treat blood type A patient having cancer.

When alpha gal containing conjugate is used to treat cancer or autoimmune diseases or other diseases, the patient can be given a vaccine that can induce/increase anti alpha-gal antibody production/efficacy (e.g. alpha gal-KLH conjugate with booster) prior and/or during the treatment. This will increase the production of anti-alpha gal antibody and increase the antibody's affinity/potency. This vaccination strategy can also be used for the method in the current invention and other known treatment methods using alpha-gal or L-rhamnose or DNP to recruiting endogenous antibody to treat disease (e.g. alpha-gal or L-rhamnose or DNP based glycol lipid for cancer treatment, alpha gal-aptamer conjugate the methods used by Centauri Therapeutics for cancer or pathogen inactivation) by giving patient alpha-gal or L-rhamnose or DNP based vaccine to boost their corresponding antibody potency prior or during the treatment. Centauri Therapeutics uses alpha gal-aptamer conjugate to treat cancer or pathogens. The aptamer has affinity to cancer cells or pathogens. The alpha-gal in Centauri Therapeutics' alpha gal-aptamer conjugate can be replaced with DNP (Dinitrophenyl) or L-rhamnose (e.g. alpha L-rhamnose) to inactivate cancer cells or pathogens.

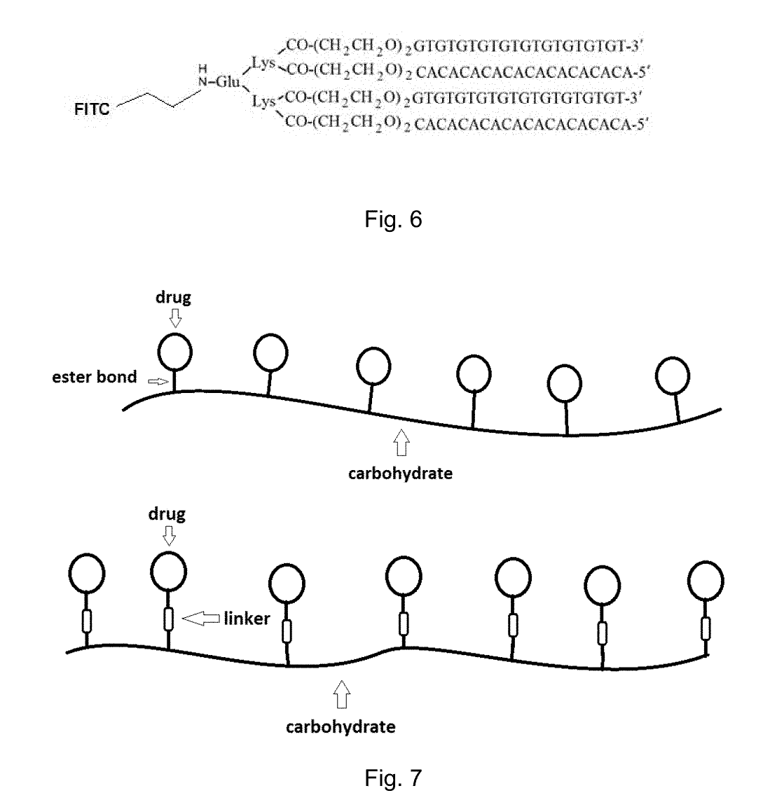

Engineered T cell therapy involving TCRs or CARS utilize T cells having TCR or CAR that can bind to a specific antigen. The patient will have these T cells in their body during and after the treatment. Once can give a patient engineered T cells (either TCR or CAR type) and then use this specific engineered T cells to attack the auto immune T cells or auto immune B cells to treat his autoimmune diseases, by give the patient the conjugate: auto antigen-(optional linker)-antigen for the engineered T cells. For example, Chimeric antigen receptor (CAR)-engineered T cells (CAR-Ts) that can recognize FITC was prepared based on the publication in J. Am. Chem. Soc., 2015, 137 (8), pp 2832-2835. When this CAR-Ts is given to the patient, conjugate FITC-auto antigen for B cell or conjugate FITC-auto antigen for T cell (MHC-peptide complex) can be given to the patient to treat his auto immune disease. One example of the conjugate is shown in FIG. 6 to treat lupus by inactivating the DNA specific auto antibody producing B cells. The patient having SLE will receive 200 mg˜1 g of the said conjugate as weekly i.v. injection to treat SLE until symptom disappear.

The method and reagent of the current invention can also be used to inactivate pathogens such as virus and bacterial when the affinity ligand against cancer cell/auto immuno T/B cell is replaced with affinity ligand against bacterial or virus (e.g. antibody/aptamer against bacterial or virus).

Examples of toxin/cell inhibitor/inactivator include but not limited to any agent that can kill the cell or inhibit the cell's normal or specific function (e.g. producing certain molecules such as protein (e.g. antibody), replication, differentiation, growth, developing into mature cell or other type of cell). They could be radioactive isotope, proteins, small molecules, siRNA, antisense molecules, enzymes and etc. Examples of them include NK cytotoxic factor, TNF such as TNF-α and TNF-β(LT), perforin, granzyme, cell apoptosis inducers, free radical generating agent, cell membrane damaging agent, toxic agent, chemotherapy agent, siRNA or antisense nucleic acid for the cell normal function, cytotoxic agent and etc. Sometimes they can be made to be in precursor type or inactive type and only become active after they bind with target cell or been taken by the target cell, e.g. the antigen-donomycin conjugate described above. Using affinity molecules coupled with cell damaging reagent is widely used in the treatment of tumor. One can readily adopt the method and principle of them for the current invention. If the cell-damaging reagent is effective only inside the cell, it normally involves a mechanism crossing the cell membrane such as endocytosis.

The current invention further discloses methods and regents to treat autoimmune diseases and allergy by applying the combination of antigen and immunosuppressive agent/drug either as a physical mixture or as synthetic conjugate or as nano/micro particles or liposome to the object/patient in need. The term nano/micro particle means the particle is in either nanometer or micrometer range of size (diameter). For example, the nano/micro particle can be in the size range of 50 nm˜50 um. List of exemplary immunosuppressive drugs can be found at “Immunosuppressive drug” article page in Wikipedia. The immunosuppressive agent/drug (immunosuppressants) suitable for the current application include but are not limited to, statins; mTOR inhibitors, such as rapamycin or a rapamycin analog; TGF-β signaling agents; TGF-β receptor agonists; TLR (toll like receptor) inhibitors; Pattern recognition receptor inhibitors; NOD-like receptors (NLR) inhibitors; RIG-I-like receptors inhibitors; NOD2 inhibitors; histone deacetylase inhibitors, such as Trichostatin A; corticosteroids; inhibitors of mitochondrial function, such as rotenone; P38 inhibitors; NF-κβ inhibitors, such as 6Bio, Dexamethasone, TCPA-1, IKK VII; adenosine receptor agonists; prostaglandin E2 agonists (PGE2), such as Misoprostol; phosphodiesterase inhibitors, such as phosphodiesterase 4 inhibitor (PDE4), such as Rolipram; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB such as TGX-221; autophagy inhibitors, such as 3-Methyladenine; aryl hydrocarbon receptor inhibitors; proteasome inhibitor I (PSI); and oxidized ATPs, such as P2X receptor blockers. Immunosuppressants also include IDO, vitamin D3, cyclosporins, such as cyclosporine A, aryl hydrocarbon receptor inhibitors, resveratrol, azathiopurine (Aza), 6-mercaptopurine (6-MP), 6-thioguanine (6-TG), FK506, sanglifehrin A, salmeterol, mycophenolate mofetil (MMF), aspirin and other COX inhibitors, niflumic acid, estriol and triptolide, siglec ligand such as sialic acid and its derivative including poly sialic acid sialic acid-lipid conjugate. In embodiments, the immunosuppressant may comprise any of the agents provided herein. The immunosuppressant can be a compound that directly provides the immunosuppressive (e.g., tolerogenic) effect on APCs or it can be a compound that provides the immunosuppressive (e.g., tolerogenic) effect indirectly (i.e., after being processed in some way after administration). Immunosuppressants, therefore, include prodrug forms of any of the compounds provided herein.

The immunosuppressant also include Heme Oxygenase-1 (HO-1) inducer such as Cobalt protoporphyrin (CoPP), protoporphyrin IX containing a ferric iron ion (Heme B) with a chloride ligand (Hemin), hematin, iron protoporphyrin or heme degradation products as well as those described in PCT/EP2015/074819. Siglecs (Sialic acid-binding immunoglobulin-type lectins) ligand such as sialic acid or its derivatives is also another type of immunosuppressant that can be used in current invention. PD-L1 is also another type of immunosuppressant that can be used in current invention. PD-L1 can effectively inhibit cytotoxic T cell. Fragment or mimic or derivative of PD-L1 that can bind with PD-1 can also be used instead. Other inhibitory ligands that can bind with inhibitory checkpoint receptor (e.g. A2AR, BTLA, CTLA-4, KIR, LAG3, TIM-3, VISTA and etc) such as B7-H3, B7-H4 can also be used instead of PD-L1. Molecule that can promote T/B reg expansion (e.g. cytokine that can stimulate T/B reg expansion such as IL-2 and TGF-β) is also another type of immunosuppressant. Different immunosuppressant can be used as a mixture and be used in combination in the current invention.

Immunosuppressants also include nucleic acids that encode the peptides, polypeptides or proteins provided herein that result in an immunosuppressive (e.g. tolerogenic) immune response. In embodiments, therefore, the immunosuppressant is a nucleic acid that encodes a peptide, polypeptide or protein that results in an immunosuppressive (e.g., tolerogenic) immune response. The nucleic acid can be coupled to synthetic nanocarrier. The nucleic acid may be DNA or RNA, such as mRNA. In embodiments, the inventive compositions comprise a complement, such as a full-length complement, or a degenerate (due to degeneracy of the genetic code) of any of the nucleic acids provided herein. In embodiments, the nucleic acid is an expression vector that can be transcribed when transfected into a cell line. In embodiments, the expression vector may comprise a plasmid, retrovirus, or an adenovirus amongst others. Nucleic acids can be isolated or synthesized using standard molecular biology approaches, for example by using a polymerase chain reaction to produce a nucleic acid fragment, which is then purified and cloned into an expression vector.

In some embodiments, the immunosuppressants provided herein are coupled to synthetic nanocarriers. In preferable embodiments, the immunosuppressant is an element that is in addition to the material that makes up the structure of the synthetic nanocarrier. For example, in one embodiment, where the synthetic nanocarrier is made up of one or more polymers, the immunosuppressant is a compound that is in addition and coupled to the one or more polymers. As another example, in one embodiment, where the synthetic nanocarrier is made up of one or more lipids, the immunosuppressant is again in addition and coupled to the one or more lipids. In embodiments, such as where the material of the synthetic nanocarrier also results in an immunosuppressive (e.g., tolerogenic) effect, the immunosuppressant is an element present in addition to the material of the synthetic nanocarrier that results in an immunosuppressive (e.g., tolerogenic) effect.

Other exemplary immunosuppressants include, but are not limited, small molecule drugs, natural products, antibodies (e.g., antibodies against CD20, CD3, CD4), biologics-based drugs, carbohydrate-based drugs, nanoparticles, liposomes, RNAi, antisense nucleic acids, aptamers, methotrexate, NSAIDs; fingolimod; natalizumab; alemtuzumab; anti-CD16, anti-CD3; tacrolimus (FK506), etc. Further immunosuppressants, are known to those of skill in the art, and the invention is not limited in this respect. Additional immunosuppressants can be found in patent and U.S. patent application Ser. No. 13/880,778, U.S. Ser. No. 14/934,135, CA 2910579, U.S. Ser. No. 13/084,662, U.S. Ser. No. 14/269,048, U.S. Pat. No. 8,652,487 and other patent application filed by Selecta Biosciences.

The current invention discloses methods and regents to treat autoimmune diseases and allergy by applying the mixture of antigen and immunosuppressive agent topically to the object/patient in need. It can also be used to inhibit the generation of anti drug antibody when the antigen is the drug (e.g. a protein drug) or its epitope. It will induce immune tolerance for the antigen. Examples of the formulation suitable for the current application include solid form such as powder, gel, lotion, ointment, solution, spray, suppository, lozenge, tablet and patch that can be topically applied to the skin or mucosa. The term topical drug delivery include drug delivery route other than injection. It includes applying drug to skin or mucosa. It includes intranasal delivery, rectal delivery, sublingual delivery and oral mucosa delivery. The immunosuppressive agent can be in the form of active agent, prodrug form, micro particle or nano particle form or liposome form. The antigen can be either B cell antigen/epitope or T cell antigen/epitope (e.g. MHC-peptide complex or conjugate; or the peptide antigen that can bind with MHC) or their combination. The combination can be either B cell antigen/epitope with T cell antigen/epitope; or the combination of several different B cell antigen/epitope and/or several different T cell antigen/epitope targeting the same disease or different diseases. The use of peptide antigen (T cell epitope) that can bind with MHC to form MHC-peptide complex in vivo (T cell antigen) instead of the peptide-MHC complex reduce the size and molecular weight, therefore improve the transdermal delivery. Examples of them can be found in the current application and related publications and patent applications.

In some embodiments, the method is to use a patch containing both antigen/allergen and immune suppressive drug (the drug listed above such as rapamycin or fujimycin or methotrexate or sialic acid or its derivative or high affinity siglec binder or their combination). The sialic acid can be either free sialic acid or sialic acid ester, sialic acid-lipid conjugate For example, sialic acid can be conjugated to cholesterol to form an ester bond using the —COOH of sialic acid with the —OH of the cholesterol. This conjugate will have better transdermal and cell membrane permeation capability. The fatty acid can also be conjugated with sialic acid's —OH to form the conjugate. These conjugate will work as immune suppressive drug after being transdermally delivered. Examples of high affinity Siglec ligands can be found in U.S. Pat. No. 8,357,671.

The transdermal or transmucosal delivery of both antigen and immunosuppressive drug will induce immune tolerance via DC cells in the skin. This would be a much easier strategy for food allergy and auto immune diseases treatment. The skin may be intact or may be exfoliated to remove stratum corneum layer to increase drug delivery. Micro needle system can also be used to the skin. The micro needle in the micro needle system can be made of bio degradable material such as PLGA encapsulating antigen and immunosuppressant. Alternatively, a bio degradable implant encapsulating antigen and immunosuppressant can also be used. The size of the implant can be bigger than 10 um in diameter, preferably >100 um, if the implant is a macro particle. For example, a 2 mm (length)×0.3 mm (diameter) rod made with PLGA containing 3 mg rapamycin and 1 mg gliadin can be used as an implant underneath the skin to treat gluten intolerance. Other implant format can also be used such as NanoPortal Capsule from Nanoprecision Medical and Medici Drug Delivery System from Intarcia, as long as they can deliver the antigen and immunosuppressant simultaneously.

DBV Technologies and other groups (e.g. those in Epicutaneous Immunotherapy for Aeroallergen and Food Allergy DOI: 10.1007/s40521-013-0003-8) are using skin patch containing allergen to treat allergy by inducing tolerance for the antigen (allergen). The topically patch or other formulation can be readily adopted for the current application. For example the topical applied formulation such as patch described in U.S. Ser. No. 15/135,914, U.S. Pat. No. 6,676,961, U.S. Ser. No. 15/111,204, U.S. Pat. No. 8,932,596B2, U.S. Ser. No. 15/184,933A1 and U.S. Pat. No. 8,202,533B2 can be adopted for the current application by adding additional immune suppressive drug in the patch (e.g. 0.1 mg-20 mg of rapamycin or fujimycin or 1 mg-100 mg methotrexate or their directives or prodrug) as well as those commercial available patch (e.g. Viaskin® Milk and Viaskin® Peanut). The administration method can be essentially the same as the prior arts except the patch contains immunosuppressants. Additional transdermal enhancer (e.g. DMSO, Azone, fatty acid, hyaluronic acid and etc, which can be found in the publication readily as well as their suitable amount) can be added to the patch or applied to the skin before applying the patch. Example of transdermal enhancing agent can be added include DMSO (e.g. 10˜300 mg/patch), azone (e.g. 1%˜10% of total drug weight), surfactant, fatty acid (e.g. 1%˜10% oleic acid). The skin can also remove for stratum corneum with be exfoliation or other means to enhance the transdermal delivery. In one example, the patch contains 500 ug-10 mg gluten (e.g. G5004 Sigma Gluten from wheat) and 0.1 mg˜10 mg of rapamycin or 1 mg-50 mg methotrexate, the gluten and rapamycin and/or methotrexate can be in powder form or film form, which can be simply mixed together physically or co-dissolved and then dried and then placed in the patch. In another example, the patch contains 5 mg gluten (e.g. G5004 Sigma Gluten from wheat) and 5 mg of rapamycin or 50 mg methotrexate and optionally additional 30 mg azone. In another example, the patch contains 5 mg gluten (e.g. G5004 Sigma Gluten from wheat) and 1 g of sialic acid or sialic acid-cholesterol conjugate either as mixture of powder or liposome form. This can be used to treat Gluten intolerance. The gluten can be replaced with gliadin instead. In embodiments, the patch can be applied daily for 1-50 weeks. In another example, the antigen is peanut antigen ara h2 200 ug and 2 mg of rapamycin is in the patch to treat peanut allergy. In one example, peanut antigen ara h2 200 ug, 2 mg of rapamycin and 50 mg sucrose is dissolved in water and then lyophilized and then placed in the patch. In one example, peanut antigen ara h2 200 ug, 2 mg of rapamycin, 50 mg SDS and 50 mg sucrose is dissolved in water and then lyophilized and then placed in the patch. In one example, peanut antigen ara h2 200 ug, 2 mg of rapamycin, 100 mg DMSO and 50 mg sucrose is dissolved in water and then lyophilized and then placed in the patch. In another example, the antigen is the double strand DNA (1 mg˜10 mg) in the previous figures to treat lupus and the drug is 3 mg of rapamycin or fujimycin or Temsirolimus. In another example, the nasal spray contains 1 mg gluten (e.g. G5004 from Sigma, Gluten from wheat) and 1 mg of rapamycin or 10 mg methotrexate in a suitable form for each spray. In another example, the sublingual lozenge contains 50 mg gluten (e.g. G5004 Sigma Gluten from wheat) and 1 mg of rapamycin or 20 mg methotrexate. In another example, the gel contains 50 mg gluten (e.g. G5004 Sigma Gluten from wheat) and 2 mg of rapamycin or 20 mg methotrexate in each 1 ml of gel. The immunosuppressant drug or the antigen or their combination can be either in the form of powder or gel or semi liquid or in the form of liposome (e.g. 100 nm˜5 um diameter) or in a nano/micro particle (e.g. 100 nm˜1 um) or being conjugated to a dendrimer or linear polymer (e.g. couple to poly acrylic acid or poly Sialic acid via ester bond to form a polymer based prodrug with MW=5K˜500K).

Other pharmaceutically acceptable amount of antigen and immunosuppressant can also be used in the patch, as long as it can produce satisfactory therapeutical (e.g. immune tolerance) effect, which can be determined experimentally by screening and testing with well-known protocol and methods.

The transdermal delivery of both antigen and immunosuppressive drug will be uptaken by APC in the skin, induce/activate tolerogenic dendritic cell and Treg/Breg, inhibit B cell activation/antibody production, germinal centre formation and antigen-specific hypersensitivity reactions, resulting in long term antigen specific immune tolerance.

A skin patch (also called transdermal patch) is a medicated adhesive patch or attachable patch that is placed on the skin to deliver a specific dose of medication through the skin and into the bloodstream. A wide variety of pharmaceuticals are now available in transdermal patch form.

There are several main types of skin/transdermal patches. The Single-layer Drug-in-Adhesive type is that the adhesive layer of this system also contains the drug. In this type of patch the adhesive layer not only serves to adhere the various layers together, along with the entire system to the skin, but is also responsible for the releasing of the drug. The adhesive layer is surrounded by a temporary liner and a backing. The Multi-layer Drug-in-Adhesive type is the multi-layer drug-in-adhesive patch is similar to the single-layer system; the multi-layer system is different, however, in that it adds another layer of drug-in-adhesive, usually separated by a membrane (but not in all cases). One of the layers is for immediate release of the drug and other layer is for control release of drug from the reservoir. This patch also has a temporary liner-layer and a permanent backing. The drug release from this depends on membrane permeability and diffusion of drug molecules. The Reservoir type is unlike the single-layer and multi-layer drug-in-adhesive systems, the reservoir transdermal system has a separate drug layer. The drug layer can be a liquid or gel or powder compartment containing a drug solution or suspension or powder separated by the adhesive layer. This patch is also backed by the backing layer. In this type of system the rate of release is zero order. The Matrix type has a drug layer of a solid or semisolid matrix containing a drug solution or suspension or solid layer such as powder or film. The adhesive layer in this patch surrounds the drug layer, partially overlaying it. In some embodiments, the reservoir type and the matrix type can be used for current invention.

In one example, antigen and immunosuppressant loaded matrix-type transdermal patch is prepared by using solvent casting method. A petri dish with a total area of 50 cm2 is used. Antigen and immunosuppressant are dissolved in 5 mL of water, methanol (1:1) solution and mixed until clear solution is obtained. 200 mg polyethylene glycol 400 is used as plasticizer and optional 100 mg propylene glycol or oleic acid or tween 80 is used as permeation enhancer, together with 100 mg sucrose they are added to the antigen/immunosuppressant solution. The resulted uniform solution is cast on the petri dish, which is lubricated with glycerin and lyophilized or dried at room temperature for 24 h. Next the dried patch is placed on a cellulose acetate membrane used as backing membrane. In another example, weighed amount of PVA (2.5% w/v) is added to a distilled water and a homogenous solution is made by constant stirring and intermittent heating at 60° C. for a few seconds and poured into glass molds already wrapped with aluminium foil around open ends and are kept for drying at 60° C. for 6 h, forming a smooth, uniform, and transparent backing membrane. Backing membrane is used as a support for antigen and immunosuppressant containing matrix.

In some embodiments, the skin patch device used in the method of the invention preferably comprises a backing, the periphery of said backing being adapted to create with the skin a hermetically closed chamber. This backing bears on its skin facing side within the chamber the composition used to decrease the skin reactivity. Preferably, the periphery of the backing has adhesive properties and forms an airtight joint to create with the skin a hermetically closed chamber.

In a particular embodiment, the composition allergens and immunosuppressants are maintained on the backing by means of electrostatic and/or Van der Waals forces. This embodiment is particularly suited where the composition allergens are in solid or dry form (e.g., particles), although it may also be used, indirectly, where the allergens are in a liquid form. Within the context of the present invention, the term “electrostatic force” generally designates any non-covalent force involving electric charges. The term Van der Waals forces designates non-covalent forces created between the surface of the backing and the solid allergen, and may be of three kinds: permanent dipoles forces, induced dipoles forces, and London-Van der Waals forces. Electrostatic forces and Van der Waals forces may act separately or together. In this respect, in a preferred embodiment, the patch device comprises an electrostatic backing. As used herein, the expression “electrostatic backing” denotes any backing made of a material capable of accumulating electrostatic charges and/or generating Van der Waals forces, for example, by rubbing, heating or ionization, and of conserving such charges. The electrostatic backing typically includes a surface with space charges, which may be dispersed uniformly or not. The charges that appear on one side or the other of the surface of the backing may be positive or negative, depending on the material constituting said backing, and on the method used to create the charges. In all cases, the positive or negative charges distributed over the surface of the backing cause forces of attraction on conducting or non-conducting materials, thereby allowing to maintain the allergen and immunosuppressant. The particles also may be ionized, thereby causing the same type of electrostatic forces of attraction between the particles and the backing. Examples of materials suitable to provide electrostatic backings are glass or a polymer chosen from the group comprising cellulose plastics (CA, CP), polyethylene (PE), polyethylen terephtalate (PET), polyvinyl chlorides (PVCs), polypropylenes, polystyrenes, polycarbonates, polyacrylics, in particular poly(methyl methacrylate) (PMMA) and fluoropolymers (PTFE for example). The foregoing list is in no way limiting.

The back of the backing may be covered with a label which may be peeled off just before application. This label makes it possible, for instance, to store the composition allergen in the dark when the backing is at least partially translucent. The intensity of the force between a surface and a particle can be enhanced or lowered by the presence of a thin water film due to the presence of moisture. Generally, the patch is made and kept in a dry place. The moisture shall be low enough to allow the active ingredient to be conserved. The moisture rate can be regulated in order to get the maximum adhesion forces. As discussed above, the use of an electrostatic backing is particularly advantageous where the allergen is in a dry form, e.g., in the form of particles. Furthermore, the particle size may be adjusted by the skilled person to improve the efficiency of electrostatic and/or Van der Waals forces, to maintain particles on the support.

In a specific embodiment, the patch comprises a polymeric or metal or metal coated polymeric backing and the particles of composition allergens are maintained on the backing essentially by means of Van der Waals forces. Preferably, to maintain particles on the support by Van der Waals forces, the average size of the particles is lower than 60 micrometer. In another embodiment, the allergens are maintained on the backing by means of an adhesive coating on the backing. The backing can be completely covered with adhesive material or only in part. Different occlusive backings can be used such as polyethylene or PET films coated with aluminium, or PE, PVC, or PET foams with an adhesive layer (acrylic, silicone, etc.). Examples of patch devices for use in the present invention are disclosed in U.S. patent application Ser. No. 11/915,926 or U.S. Pat. No. 7,635,488.

Other examples are disclosed in U.S. Ser. No. 13/230,689, which also discloses a spray-drying process to load the substance in particulate form on the backing of a patch device. An electrospray device uses high voltage to disperse a liquid in the fine aerosol. Allergens and immunosuppressants dissolved in a solvent are then pulverized on the patch backing where the solvent evaporates, leaving allergens and immunosuppressants in particles form. The solvent may be, for instance, water or ethanol, according to the desired evaporation time. Other solvents may be chosen by the skilled person. This type of process to apply substances on patch backing allows nano-sized and mono-sized particles with a regular and uniform repartition of particles on the backing. This technique is adapted to any type of patch such as patch with backing comprising insulating polymer, doped polymer or polymer recovered with conductive layer. Preferably, the backing comprises a conductive material.

In another embodiment, the periphery of the backing is covered with a dry hydrophilic capable of forming an adhesive hydrogel film by contact with the moistured skin (as described in U.S. Ser. No. 12/680,893). In this embodiment, the skin has to be moistured before the application of the patch. When the hydrogel comes into contact with the moistured skin, the polymer particles absorb the liquid and become adhesive, thereby creating a hermetically closed chamber when the patch is applied on the skin. Examples of such hydrogels include polyvinylpyrolidone, polyacrylate of Na, copolymer ether methyl vinyl and maleic anhydride.

In another particular embodiment, the liquid composition allergen and immunosuppressant is held on the support of the patch in a reservoir of absorbent material. The composition may consist in an allergen +immunosuppressant solution or in a dispersion of the mixture, for example in glycerine. The adsorbent material can be made, for example, of cellulose acetate.

The backing may be rigid or flexible, may or may not be hydrophilic, and may or may not be translucent, depending on the constituent material. In the case of glass, the support may be made break-resistant by bonding a sheet of plastic to the glass. In one embodiment, the backing of the patch contains a transparent zone allowing directly observing and controlling the inflammatory reaction, without necessarily having to remove the patch. Suitable transparent materials include polyethylene film, polyester (polyethylene-terephtalate) film, polycarbonate and every transparent or translucent biocompatible film or material.

FIG. 7 shows the examples of drug conjugated to carbohydrate polymer to form prodrug. The novel prodrugs can be in the form of carbohydrate (or other polymer) drug conjugate in which the drug is conjugated to the carbohydrate (or other polymer) with cleavable linkage. More than one drugs can be conjugated to the polymer backbone. Suitable carbohydrate includes sialic acid containing polymer, hyaluronic acid, chondroitin sulfate, dextran, carboxyl dextran, cellulose, carboxyl cellulose and their derivatives. In some embodiments, preferably the carbohydrate is selected from sialic acid containing polymer, hyaluronic acid, starch, dextran and chondroitin sulfate. Preferably the drug is conjugated to the polymer backbone with biodegradable linker or bond such as ester bond or a linker containing ester bond. Examples of the ester bond can be that formed between the —COOH group of the carbohydrate and the —OH of the drug or that formed between the —OH group of the carbohydrate and the —COOH of the drug if the drug, contains —COOH group. For example, the average MW of the carbohydrate or other polymer carrier is between 1K˜100K. The carbohydrate is a long chain polymer. After conjugation, it will carry multiple drugs. Preferably the number of the drug conjugated is >5 on each carbohydrate molecule or other polymer backbone. In some embodiments the number of the drug conjugated is >10 on each polymer backbone.

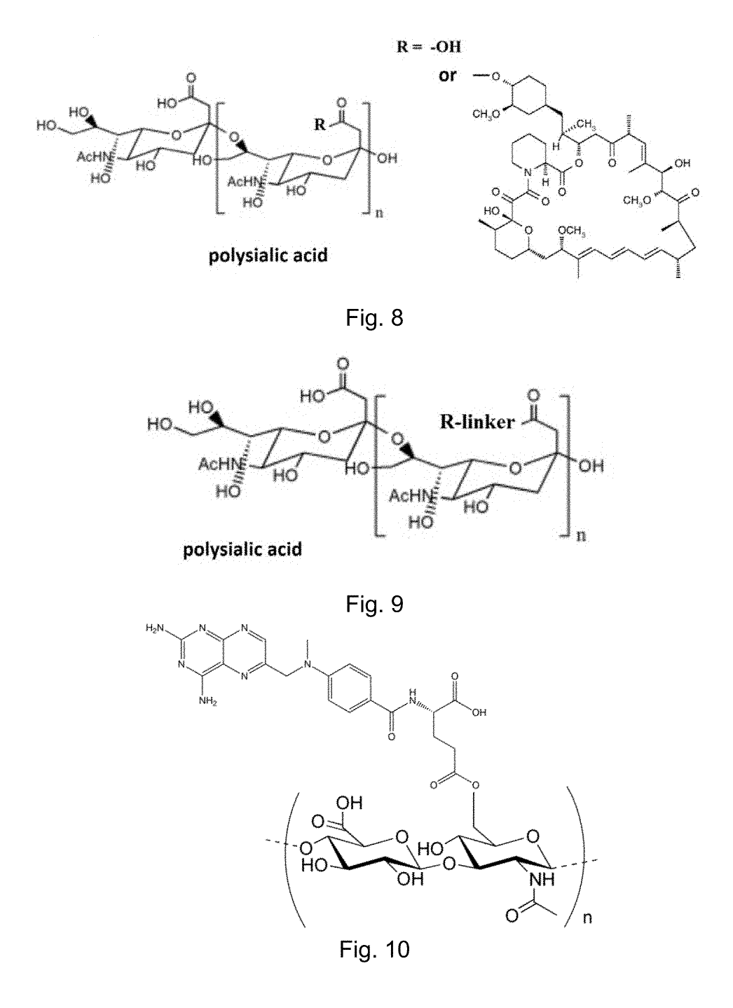

The sialic acid containing polymer suitable for the current invention include poly sialic acid formed by sialic acid monomer connected with α2,3 or α2,6 or α2,8 or α2,9 linkage or their combination. It also includes graft polymer or branched polymer containing sialic acid. It can also be a linear polymer backbone dextran or synthetic polymer such as PVA, PAA). FIG. 8 shows an example of polysialic acid conjugated with rapamycin with ester bond to form a prodrug. The R can also be replaced with lipid type molecule to be used in nano/micro particle encapsulation or used in liposome as described above. A linker can also be added between the drug and polymer backbone, FIG. 9 shows an example in which the linker is a glycine. The drug R in FIG. 9 can be rapamycin or other immuno suppressive drug. FIG. 10 shows an example of hyaluronic acid based methotrexate prodrug.

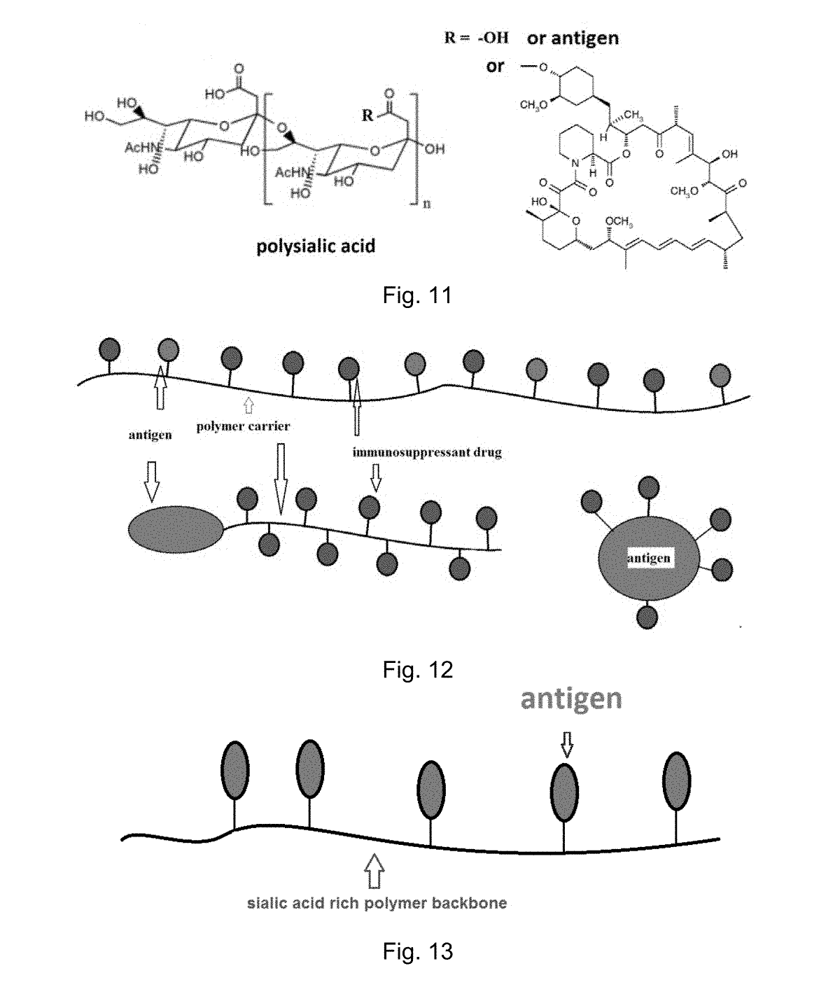

Furthermore, the immune suppressive drug can also be directly conjugated to antigen or conjugated to the antigen via a linker or carrier and used in the patch. The carrier can be a polymer. For example, the poly sialic acid-rapamycin in FIG. 8 can be used to conjugate to the protein's lysine with EDC coupling (e.g. gluten or antibody drug or gliadin or is peanut antigen protein ara h2) and be used in the patch (e.g. 100 ug˜15 mg) instead of the mixture of antigen and drug. FIG. 11 shows an example of the structure of the conjugate containing both antigen and rapamycin.

Other chemistry such as maleimide —SH coupling can also be used to conjugate drug with antigen (via linker or polymer carrier). The general structure of the conjugate is antigen-drug or antigen-linker drug or antigen-carrier-drug. More than one drug molecule can be conjugated to each antigen. If the polymer carrier is used, the resulting conjugate can contain multiple antigen and multiple drug molecules. In some embodiments, the number of antigen molecule in each polymer is less than 6 to avoid complement activation if the antigen is B cell antigen. The FIG. 12 shows examples of 3 different formats of the antigen-drug conjugate.

When liposome is used, either the drug or both the antigen and immune suppressive drug can be encapsulated in the liposome.

Dendritic cell is abundant in skin, adding DC regulating drug with antigen/allergen in a patch can be effective to induce tolerance.

Besides being applied topically, the mixture or conjugate can also be injected or taken orally to induce immune tolerance and to treat auto immune disease/allergy.

The topical formulation or implant can contain either antigen+drug or antigen-drug conjugate or encapsulated antigen/drug (e.g. in microsphere or liposome) or their combinations. The antigen can be either in the form of crude antigen (e.g. peanut extract, gluten) or purified antigen (e.g. peanut antigen protein ara h2, gliadin) or antigen-drug conjugate or encapsulated antigen (e.g. in microsphere or liposome) or their mixture.

In another format, as shown in FIG. 13, the Epitope(antigen)-Sialic acid rich polymer conjugate or Epitope(antigen)-Siglec ligand rich polymer conjugate can be used to treat autoimmune disease or allergy or to induce immune tolerance, which can be either injected or implanted (being encapsulated inside the implant) or applied topically. The pharmaceutically acceptable amount of conjugate can also be used, as long as it can produce satisfactory therapeutic (e.g. immune tolerance) effect, which can be determined experimentally by screening and testing with well-known protocol.

The term Sialic acid rich polymer means a polymer having multiple sialic acids or siglec ligand conjugated to its back bone. The back bone can be a branched or linear polymer or dendrimer such as synthetic polymer PVA, PAA, polyamine, or nature polymer such as polysialic acid, carbohydrate. The sialic acid or sialic acid containing fragments or siglec ligands are conjugated to the polymer back bone. Sialic acid polymer contains either α2,3 or α2,6 or α2,8 sialoside or sialic acid or their derivatives (e.g. those described in J Immunol. 2006 Sep. 1; 177(5):2994-3003, U.S. Pat. No. 9,522,183 and U.S. Pat. No. 8,357,671) that can bind with Siglec. The oligo/poly sialic acid with α2,8 linkage backbone itself is also a sialic acid rich polymer. The sialic acid rich polymer can also contains the mixture of different sialoside, sialic acid and/or their derivatives on its backbone. The liposome having sialic acid or sialoside attached on its surface can also be regarded as a sialic acid rich polymer (e.g. those described in U.S. Pat. No. 9,522,183).

There are many sialic acid/siglec ligand rich polymer suitable for the current application can be readily found in the literature, for example, those described in J Immunol. 2006 Sep. 1; 177(5):2994-3003, Nat Chem Biol. 2014 January; 10(1):69-75, J Am Chem Soc. 2013 Dec. 11; 135(49):18280-18283, J Immunol. 2014 Nov. 1; 193(9):4312-21, J Allergy Clin Immunol. 2017 January; 139(1):366-369.e2, Angew Chem Int Ed Engl. 2015 Dec. 21; 54(52):15782-8, Proc Natl Acad Sci U S A. 2009 Feb. 24; 106(8):2500-5, J Exp Med. 2010 Jan. 18; 207(1):173-87, J Immunol. 2013 Aug. 15; 191(4):1724-31, Proc Natl Acad Sci U S A. 2016 Sep. 13; 113(37):10304-9, J Clin Invest. 2013 July; 123(7):3074-83, Proc Natl Acad Sci U S A. 2016 Mar. 22; 113(12):3329-34, U.S. Pat. No. 9,180,182 and U.S. Pat. No. 9,552,183. These sialic acid/siglec ligand rich polymers can be readily adopted for the current inventions. In some embodiments each polymer is conjugated with less than 6 copies of antigen when B cell antigen is used to reduce the risk of compliment activation, in some embodiments each polymer is conjugated to only one antigen. In some embodiments each polymer is conjugated with more than 10 copies of antigen.

Using epitope (antigen)-sialic acid rich polymer conjugate, the antigen will bind with the auto immune T cell or B cell clones, which will guide the conjugated sialic acid rich polymer to inactivate these antigen specific auto immune T cell or B cell clones selectively.

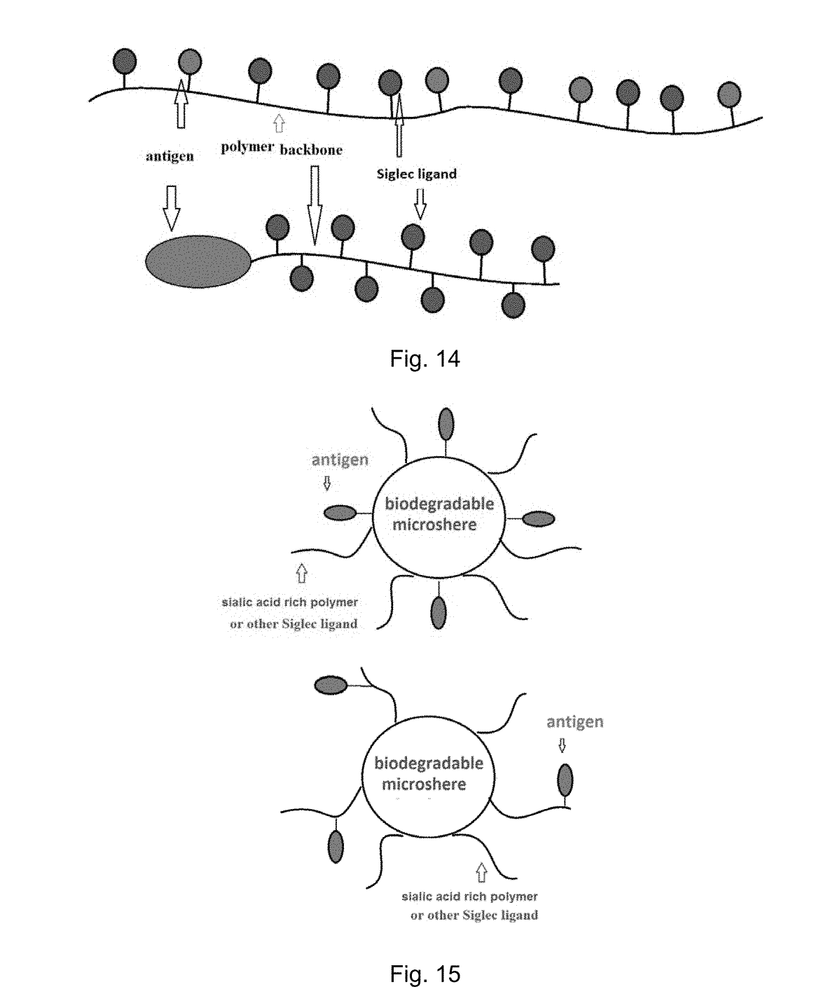

FIG. 14 shows examples of the conjugate containing sialic acid/siglec ligand suitable for the current inventions. Optional linkers can be added between the antigen and the polymer and/or between siglec ligand and the polymer.

When liposome expressing both antigen and siglec ligand is used (e.g. those described in the current invention and those in J Clin Invest. 2013 July; 123(7):3074-83, J Immunol. 2013 Aug. 15; 191(4):1724-31 and U.S. Pat. No. 9,552,183), the liposome can further encapsulate immuno suppressive drug such as rapamycin. For example, each liposome particle can contain pharmaceutical effective amount of rapamycin (e.g. 1%˜50% liposome weight of rapamycin). This will further increase the efficacy to induce immuno tolerance and treating auto immune diseases/allergy.

Another format suitable for the current application is to use microsphere. The term microsphere include particles from nano meter size to micrometers (e.g. 50 nm˜5 um in diameter). Preferably the microsphere is bio degradable (e.g. made of biodegradable polymer such as poly(lactidecoglycolide)(PLGA)), the microsphere can further encapsulate immune suppressive drug such as rapamycin (e.g. 1%˜80% weight of the microsphere).

FIG. 15 shows schematic examples of the structure of the microsphere based agent to induce immune tolerance and treating auto immune diseases/allergy. For example, the microsphere can be biodegradable synthetic polymer such as PLGA. Immune suppressive drug such as rapamycin (e.g. 1%˜80% weight of the microsphere) is encapsulated. The size of the microsphere is 3 um or 300 nm. Sialic acid rich polymer or other siglec ligand is conjugated to the surface of the microsphere directly or with a linker, antigen is also conjugated to the surface of the microsphere directly or with a linker. Alternatively, the Sialic acid rich polymer is conjugated to the surface of the microsphere directly or with a linker and the antigen is conjugated to the Sialic acid rich polymer. The antigen can also be encapsulated in the microsphere as well. Alternatively, the drug (immunosuppressant) can be conjugated to the surface of the microsphere or conjugated to the sialic acid rich polymer instead of being encapsulated. Examples of microsphere suitable for the current application can be readily adopted from the disclosure in the publications such as those in U.S. patent application Ser. No. 13/880,778, U.S. Ser. No. 14/934,135, CA 2910579, U.S. Ser. No. 13/084,662 and U.S. Pat. No. 8,652,487 and other patent application filed by Selecta Biosciences. It can be used to treat autoimmune disease or allergy or to induce immune tolerance, which can be either injected or implanted (being encapsulated inside the implant) or applied topically to the patient. The pharmaceutically acceptable amount of these types of conjugate can also be used, as long as it can produce satisfactory therapeutical (e.g. immune tolerance) effect, which can be determined experimentally by screening and testing with well-known protocol.

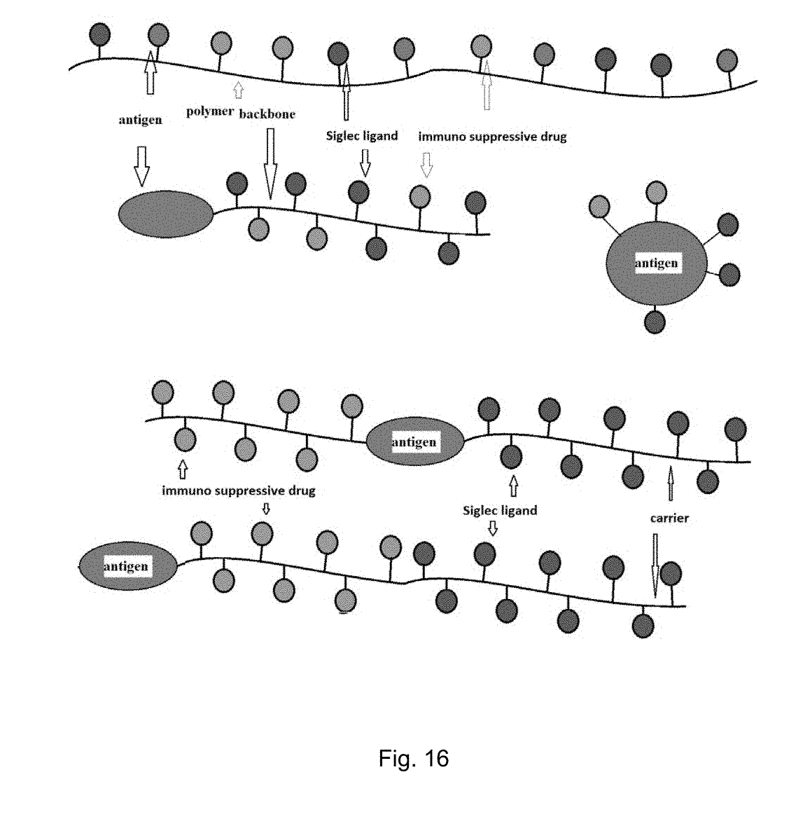

Another format suitable for the current application is to use polymer carrier conjugated with antigen, siglec ligand and/or other immunosuppressant, which is shown in the FIG. 16. Alternatively, both siglec ligand and other immunosuppressant can be conjugated to the antigen. The FIG. 16 shows different formats suitable for the current invention. The polymer conjugated with multiple antigen (e.g. 1-100), multiple siglec ligands (e.g. 5˜500 copies) and multiple copies of other immunosuppressant is essentially the previous described polymer conjugated with antigen and siglec ligand, which is further conjugated with multiple immunosuppressant molecules (e.g. 5˜500 molecules). Alternatively the polymer conjugated with multiple immunosuppressant molecules and multiple siglec ligands can be conjugated to one antigen molecule. Alternatively, multiple immunosuppressant molecules and multiple siglec ligands can be conjugated to one antigen molecule directly or with linker but without polymer carrier. Alternatively, one or more polymer conjugated with multiple immunosuppressant molecules and one or more multiple polymer conjugated with siglec ligands can be conjugated to one antigen molecule. Alternatively, one or more polymer conjugated with multiple immunosuppressant molecules and one or more multiple polymer conjugated with siglec ligands can be conjugated together and then conjugated to one antigen molecule.

They can be used to treat autoimmune disease or allergy or to induce immune tolerance caused by the antigen used to construct these conjugate, which can be either injected or implanted (being encapsulated inside the implant) or applied topically to the subject in need. The pharmaceutically acceptable amount of conjugate in pharmaceutically acceptable formulation can be used, as long as it can produce satisfactory therapeutical (e.g. immune tolerance) effect, which can be determined experimentally by screening and testing with well-known protocol. This method can be used to treat antigen specific autoimmune disease or allergy.

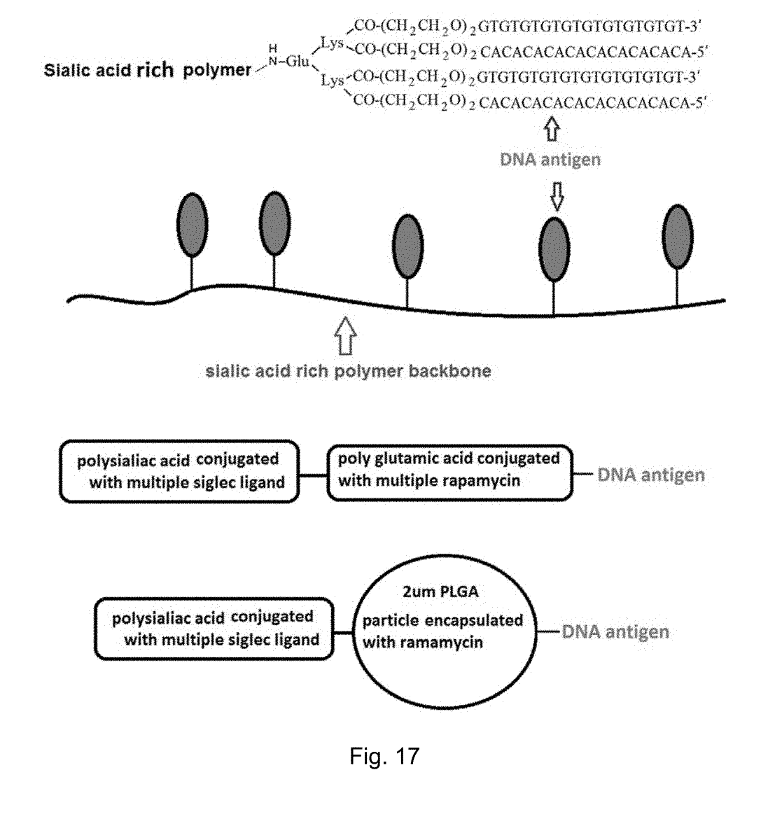

Examples of Sialic acid rich polymer-Antigen conjugate for systemic lupus erythematosus are shown in the FIG. 17. The sialic acid polymer-Antigen conjugate for SLE treatment has the structure of DNA-linker-Sialic acid polymer. In one example, the patient having SLE will receive 200 mg˜1 g of the said conjugate as weekly i.v. injection to treat SLE.

The above transdermal delivery system using the combination of antigen and immune suppressant agent are used for allergy, autoimmune diseases and antidrug antibody treatment. When the immune suppressant agent in the above example and methods is replaced with immune enhancing agent (e.g. vaccine adjuvant such as TLR agonist) and the antigen is a pathogen antigen, the transdermal delivery system becomes a vaccine or booster for the pathogen antigen. For example, the transdermal delivery system is a skin patch containing co-formulated immune enhancing agent together with pathogen antigen with optional transdermal delivery enhancer (e.g. azone, fatty acid, hyaluronic acid) in Viaskin® patch or similar dermal patch. It can also be a lotion, gel, liquid, spray, film or other dosage form suitable for topically applied to the skin or membrane. Vaccine adjuvant type molecule such as TLR agonists can be used in the current invention such as MPLA, CpG ODNs, imiquimod, poly IC, resiquimod, gardiquimod, R848 and 3M-052. Examples of the antigen can be either synthetic or purified or the mixture made of pathogen. For example, it can be HIV gp-120, it can be flu it can be the flu virus lysate, it can be HBV surface antigen and it can be tumor cell lysate. Using these antigens will generate immune response against the pathogen as a vaccine or booster.

In some embodiments, the topical formulations contain 0.1˜100 mg antigen, 0.1˜50 mg TLR agonist in each patch or each mL of gel/lotion/liquid. Transdermal enhancing agent can be added to it as well such as DMSO, azone (e.g. 1%˜10%), surfactant, fatty acid (e.g. 1%˜10% oleic acid). In one example, the formulations contain 10 mg/mL Flu virus lysate, 5 mg/mL imiquimod, 20 mg/mL SDS in 1× PBS and 5% sucrose and then being lyophilized. The lyophilized powder can be used to prepare a skin patch and attached to the skin at 10˜500 mg powder/patch. In another example, 10˜100 mg HBV surface antigen, 5-50 mg of imiquimod is mixed together and added to a Viaskin® like dermal patch. It can be applied to the skin twice every week for 2 weeks, each time for 2 day as a vaccine and then applied for 2 days as a booster after 1 month and 3 month to generate immunity against HBV. In another example, 100 mg pathogen antigen, 20 mg of poly IC, 20 mg of imiquimod and 100 mg of DMSO is mixed together and added within a skin patch. It can be applied to the skin twice every week for 2 weeks, each time for 2 day as a vaccine and then applied for 2 days as a booster after 1 month and 3 month to generate immunity against said pathogen. The pathogen antigen can be the antigen peptide that can bind with MHC to form MHC-peptide complex. Using antigen peptide instead of MHC-peptide complex improves transdermal delivery.

Compounds described herein can be administered as a pharmaceutical or medicament formulated with a pharmaceutically acceptable carrier. Accordingly, the compounds may be used in the manufacture of a medicament or pharmaceutical composition. Pharmaceutical compositions of the invention may be formulated as solutions or lyophilized powders for parenteral administration. Powders may be reconstituted by addition of a suitable diluent or other pharmaceutically acceptable carrier prior to use. Liquid formulations may be buffered, isotonic, aqueous solutions. Powders also may be sprayed in dry form. Examples of suitable diluents are normal isotonic saline solution, standard 5% dextrose in water, or buffered sodium or ammonium acetate solution. Such formulations are especially suitable for parenteral administration, but may also be used for oral administration or contained in a metered dose inhaler or nebulizer for insufflation. Compounds may be formulated to include other medically useful drugs or biological agents. The compounds also may be administered in conjunction with the administration of other drugs or biological agents useful for the disease or condition to which the invention compounds are directed.

As employed herein, the phrase “an effective amount,” refers to a dose sufficient to provide concentrations high enough to impart a beneficial effect on the recipient thereof. The specific therapeutically effective dose level for any particular subject will depend upon a variety of factors including the disorder being treated, the severity of the disorder, the activity of the specific compound, the route of administration, the rate of clearance of the compound, the duration of treatment, the drugs used in combination or coincident with the compound, the age, body weight, sex, diet, and general health of the subject, and like factors well known in the medical arts and sciences. Various general considerations taken into account in determining the “therapeutically effective amount” are known to those of skill in the art and are described. Dosage levels typically fall in the range of about 0.001 up to 100 mg/kg/day; with levels in the range of about 0.05 up to 10 mg/kg/day are generally applicable. A compound can be administered parenterally, such as intravascularly, intravenously, intraarterially, intramuscularly, subcutaneously, or the like. Administration can also be orally, nasally, rectally, transdermally or inhalationally via an aerosol. The compound may be administered as a bolus, or slowly infused. A therapeutically effective dose can be estimated initially from cell culture assays by determining an IC50. A dose can then be formulated in animal models to achieve a circulating plasma concentration range that includes the IC50 as determined in cell culture. Such information can be used to more accurately determine useful initial doses in humans. Levels of drug in plasma may be measured, for example, by HPLC. The exact formulation, route of administration and dosage can be chosen by the individual physician in view of the patient's condition.

In the current application, the “/” mark means either “and” or “or”. Unless otherwise defined, all technical and scientific terms used herein have the same meaning as commonly understood by one of ordinary skill in the art to which this invention belongs. All patents and publications mentioned in this specification are indicative of the level of those skilled in the art to which the invention pertains. All patents and publications are herein incorporated by reference to the same extent as if each individual publication was specifically and individually indicated to be incorporated by reference. The inventions described above involve many well-known chemistry, instruments, methods and skills. A skilled person can easily find the knowledge from text books such as the chemistry textbooks, scientific journal papers and other well-known reference sources.

1. A transdermal patch to treat conditions selected from autoimmune disease, allergy and anti drug antibody comprising an antigen causing said condition and an immunosuppressant.

2. The transdermal patch according to claim 1, wherein the antigen is B cell antigen.

3. The transdermal patch according to claim 1, wherein the antigen is T cell antigen in MHC-peptide complex form.

4. The transdermal patch according to claim 1, wherein the antigen is peptide of T cell antigen that can form MHC-peptide complex.

5. The transdermal patch according to claim 1, wherein the immunosuppressant is selected from rapamycin, fujimycin and methotrexate.

6. A method for treating conditions selected from autoimmune disease, allergy and anti drug antibody in a subject, comprising administering to the subject a transdermal patch according to claim 1.

7. A conjugate to treat conditions selected from autoimmune disease, allergy and anti drug antibody comprising an antigen causing the condition, a first immunosuppressant and a second immunosuppressant.

8. The conjugate according to claim 7, wherein the auto antigen is B cell antigen.

9. The conjugate according to claim 7, wherein the auto antigen is T cell antigen in MHC-peptide complex form.

10. The conjugate according to claim 7, wherein the first immunosuppressant is selected from rapamycin, fujimycin and methotrexate.

11. The conjugate according to claim 7, wherein the first immunosuppressant is selected from siglec ligand.


SEQUENCE LISTING (2 sequences)

SEQ ID NO: 1 - DNA, 20 bp, Artificial Sequence
  synthetic artificial DNA antigen
gtgtgtgtgtgtgtgtgtgt

SEQ ID NO: 2 - DNA, 20 bp, Artificial Sequence
  synthetic artificial DNA antigen
cacacacacacacacacaca